(19) United States
(12) Patent Application Publication
(10) Pub. No.: US 2010/0253906 A1
(43) Pub. Date: Oct. 7, 2010

(54) METHOD FOR MEASURING AND VERIFYING THE CORRECT ADAPTATION OF LENSES TO FRAME

(76) Inventors: Dan KATZMAN, Givat Ela (IL); Gabby Weinrot, Kibbutz Shamir (IL); Niv Adut, Kibbutz Shamir (IL)

Correspondence Address: Pearl Cohen Zedek Latzer, LLP, 1500 Broadway, 12th Floor, New York, NY, 10036, US

(21) Appl. No.: 12/419,782

(22) Filed: Apr. 7, 2009

(51) Int. Cl. A61B 3/02

(52) U.S. Cl. 351/204; 351/246

(57) ABSTRACT

A device and method for verifying the fitting of ophthalmic lenses in a frame for spectacles, the device comprising a mask to be placed on at least one lens of the spectacles, the mask having at least one transparent stain within an opaque area of the mask, the stain located correspondingly to a test point on the lens. The method comprising placing a mask on at least one lens installed in the spectacle's frame, the mask having at least one transparent stain within an opaque area of the mask, the stain located correspondingly to a test point on the lens. Additionally, a system and method for optometric measurements by using the described mask are also provided.

### BACKGROUND OF THE INVENTION

Many ophthalmic lenses, for example, lenses which are of complex design like aspheric or multifocal lenses, require exact fitting of the lens in the frame of the spectacles, so that at least some key points on the lens will be in certain positions relative to the center of the eye's pupil of the wearer. For example, one specific point should be exactly in front of the center of the pupil when the wearer looks straight forward to the horizon. This point is usually called a fitting point. Additionally, since users of multifocal lenses usually have to lower their gaze in order to look at close objects, a specific spot on the lens should be in front of the pupil when the wearer lowers his gaze.

In order to ensure the correct positions of said key points, the standard practice in the industry is to mark these points with marking pen on demo lenses installed in the chosen spectacles frame. After taking geometric measurements of these markings relative to the frame, the manufactured lens is fitted in the frame accordingly.

Usually, lens that has specific fitting conditions may have markings which may be invisible to the wearer. Those markings may enable identification of the points of interest, for example, the fitting point. An optician may visually check that those points are in the right position relative to the person's pupil when the spectacles are worn by the person. This process is based on assessment of the right position by the optician, and although the optician may use advanced vision tools for the assessment, this process may introduce an undetected error in the fitting of the lens, which may cause un-adaptation and rejection of the lens.

For example, reference is made to FIGS. 1A, 1B and 1C, which are schematic illustrations of an optical system 100 which may represent the standard method to verify the correct fitting of a lens in a frame. Optical system 100 may include a patient's pupil 10, a marked point 14 on lens 12 and an optician's pupil 16. In order to perform the examination, for example, of the fitting point, the patient may be asked to look straight forward and to hold his head in a natural position for looking straight forward, for example, at a far object. The optician may intend to check whether marked point 14 is in the right position by looking straight at patient's pupil 10 from a position in which it seems to the optician that his pupil 16 is exactly opposite to patient's pupil 10. Then, the optician may have to estimate whether patient's pupil 10, marked point 14 and his pupil 16 are all located on one straight line. In other words, marked point 14 should seem to the optician exactly in the middle of patient's pupil 10.

FIG. 1A illustrates one type of error which may be introduced by the assessment process described above. Arrow A illustrates a range of locations of optician's pupil 16 in which patient's pupil 10, marked point 14 and optician's pupil 16 may seem to the optician as located on one horizontal straight line, and the optician may deduce that marked point 14 is in the right location, although marked point 14 may not be in the right location. In principle, a correct assessment of the location of marked point 14 may be performed if the location of optician's pupil 16 is exactly opposite to patient's pupil 10, e.g., substantially in the middle of range A. Then, if patient's pupil 10, marked point 14 and optician's pupil 16 are on one straight line, it may be deduced that marked point 14 is in the correct location.

FIG. 1B illustrates another type of error which may be introduced by the assessment process described above. Arrow B illustrates a range of locations of patient's pupil 10 in which patient's pupil 10, marked point 14 and optician's pupil 16 may seem to the optician as located on one straight line, and the optician may deduce that marked point 14 is in the right location, although marked point 14 may not be in the right location.

The two types of errors which are described above with reference to FIGS. 1A and 1B, may be joined to a greater combined error, as shown in FIG. 1C.

Therefore, a method for verifying the correct adaptation of lenses to frame which provide better accuracy is needed.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B and 1C are schematic illustrations of an optical system which may represent the standard method to verify the correct fitting of a lens in a frame;

FIG. 2 is a schematic illustration of an optical system which may represent a method for verifying the correct fitting of a lens in a frame according to some embodiments of the present invention;

FIG. 3 is a schematic illustration of a system for verifying the correct fitting of a lens in a frame according to some embodiments of the present invention;

FIG. 4 is a flowchart illustrating a method for verifying the correct fitting of a lens in a spectacles frame according to some embodiments of the present invention;

FIG. 5 is a flowchart illustrating a method for verifying the correct fitting of a lens in a spectacles frame according to some embodiments of the present invention;

FIG. 6 is a schematic illustration of a system for back vertex distance and/or eye length measurements according to some embodiments of the present invention; and

FIG. 7 is a flowchart illustrating a method for optometrical measurements according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Embodiments of the present invention may provide a method and device verifying the correct fitting of a lens in a frame, which may provide much better accuracy than the standard known methods. The better accuracy may be provided by reducing the number of variables in the optical system which may be source of errors. While in the known methods the light rays corresponding to the points of interest are blocked at the points of interest, for example, by opaque markings, in embodiments of the present invention the light rays corresponding to the points of interest are allowed to pass through the lens while other rays of light in an area around the points of interest are blocked. In a method according to embodiments of the present invention the wearer of the spectacles may determine by himself if the lens are installed properly in the frame. Therefore, the assessment of a third party, e.g., the optician, may not be required.

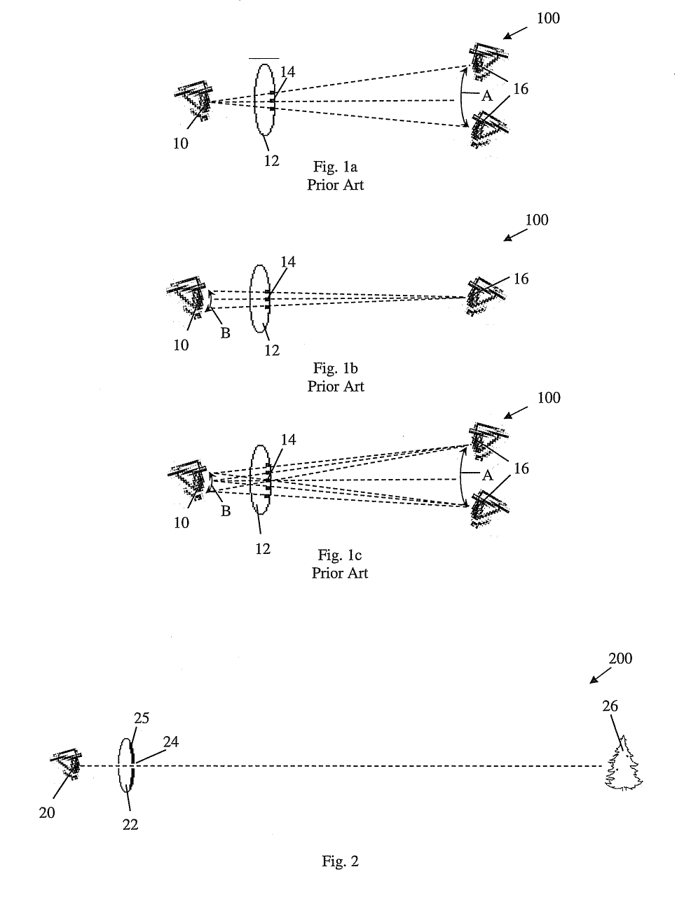

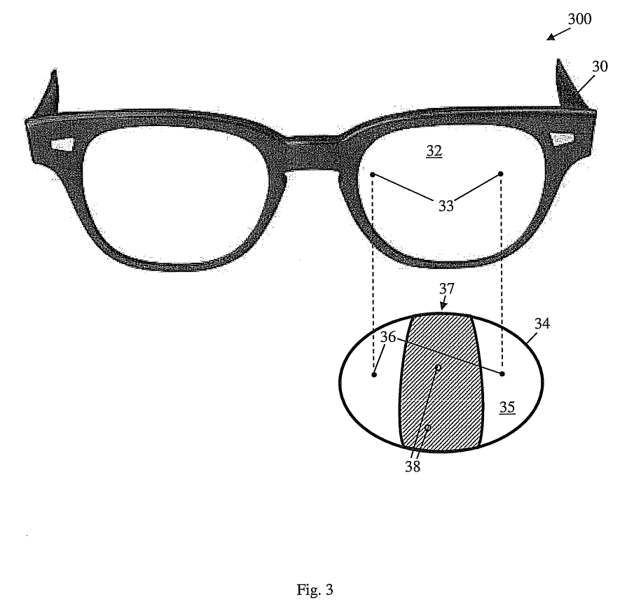

Reference is now made to FIG. 2, which is a schematic illustration of an optical system 200 which may represent a method for measuring and/or verifying the correct fitting of a lens in a frame according to some embodiments of the present invention. System 200 may include a patient pupil 20 of a patient wearing spectacles, a lens 22 which may be fitted in the spectacles frame (shown in FIG. 3), an at least partially opaque mask 25, a transparent stain 24 within an opaque area of the mask, and an object of interest 26.

Transparent stain 24 may be located on mask 25 correspondingly to a test point on the lens, as shown and described in detail below with reference to FIG. 3. That is, Opaque mask 25 may be placed on lens 22 so that transparent stain 24 is located substantially on a test point on lens 22. The test point may be the fitting point described above or a point on the lens which should be substantially in front of the pupil when the wearer lowers his gaze, or any other suitable test point. Object of interest 26 may correspond to the test point, for example, accordingly, at the test point lens 22 may have a lens power suitable for looking at object 26 by the patient. In some embodiments of the present invention, if the test point is the fitting point of lens 22, object of interest 26 is a far object so that, for example, the wearer's gaze may have to be aimed substantially to the horizon in order to see object 26.

In order to perform the examination of the adaptation of the lenses to the spectacles frame, a patient wearing spectacles in which lens 22 is fitted may look through mask 25, while the person may hold his head in a natural position for looking at object of interest 26. In case the lens is fitted correctly, transparent stain 24 should be substantially in front of pupil 20, thus, for example, enabling the spectacles wearer to see object 26.

According to different embodiments of the present invention, transparent stain 24 may have a shape of a spot, line or any other shape of interest.

According to some embodiments of the present invention. Mask 25 may be placed on lens 22 by sticking mask 25 on lens 22. For example, mask 25 may be any suitable kind of a sticker. For example, in some embodiments mask 25 may be stick to lens 22 by electrostatic effect. Mask 25 and lens 22 may include corresponding markings (for example, as described below and shown in FIG. 3), for example, in order to verify accurate positioning of mask 25 on lens 22, e.g., so that stain 24 may be located substantially on a test point on lens 22. For example, the markings on lens 22 may substantially coincide with corresponding markings on mask 25 when mask 25 is positioned accurately on lens 22.

Reference is now made to FIG. 3, which is a schematic illustration of a system 300 for measuring and/or verifying the correct fitting of a lens 32 in a frame according to some embodiments of the present invention. System 300 may include spectacles frame 30 with a lens 32 fitted therein, and a mask 34. Mask 34 may include one or more transparent area 35 and an opaque area 37. Alternatively, in some embodiments, opaque area 37 may occupy the surface of mask 34 completely. Mask 34 may further include at least one transparent stain 38 within opaque area 37, located correspondingly to a respective test point (not shown) on lens 32.

According to different embodiments of the present invention, transparent stain 38 may have a shape of a spot, line or any other shape of interest.

According to some embodiments of the present invention. Mask 34 may be placed on lens 32 by adhering mask 34 on lens 32. For example, mask 34 may be any suitable kind of a sticker. For example, in some embodiments mask 34 may be adhered to lens 32 by electrostatic effect. Mask 34 may include markings 36 which may correspond to markings 33 on lens 32, for example, in order to verify accurate positioning of mask 34 on lens 32, e.g., so that transparent stain 38 may be located substantially on the test point on lens 32. For example, the markings 33 on lens 32 may substantially coincide with corresponding markings 36 on mask 34 when mask 34 is positioned accurately on lens 32. Markings 33 may be made on lens 32 in its production process and/or may be invisible to the wearer of lens 32. However, markings 33 may be recognized, for example, highlighted, in order that markings 36 of mask 34 may be positioned substantially on them.

When mask 34 is positioned accurately on lens 32, e.g., so that transparent stain 38 may be located substantially on the test point on lens 32, a patient may wear spectacles 30 and aim his gaze in the direction of an object of interest, for example, which corresponds to the test point on lens 32, while holding his/her head in a natural position for looking at the object. In case the patient is able to see the object, he may conclude that the test point and the corresponding transparent stain 38 are located substantially in the correct position in front of his pupil, and therefore, he may conclude that lens 32 is fitted correctly in spectacles frame 30. However, in the case the patient is not able to see the object, he may conclude that the test point and the corresponding transparent stain 38 are not located in the correct position in front of his pupil and therefore, he may conclude that lens 32 is not fitted correctly in spectacles frame 30.

According to different embodiments of the present invention, transparent stain 38 may have a shape of a spot, line or any other shape of interest.

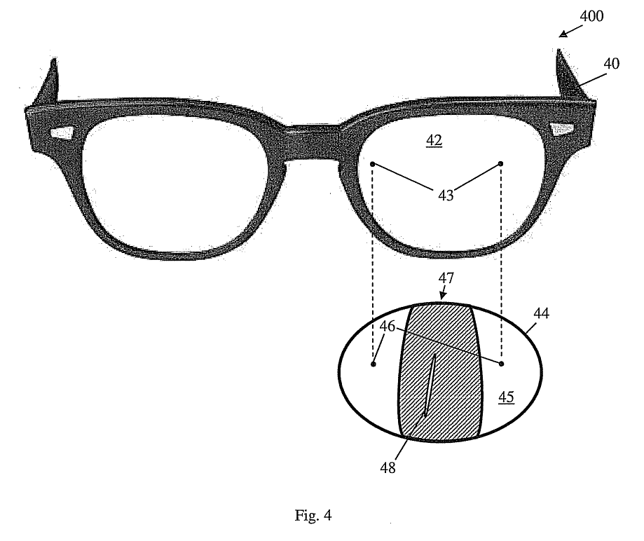

Reference is now made to FIG. 4, which is a schematic illustration of a system 400 for measuring and/or verifying the correct fitting of a multifocal lens 42 in a frame according to some embodiments of the present invention. The elements of system 400 are similar and function similarly to the corresponding elements of system 300 in FIG. 3. In some embodiments, for example, the shape of stain 48 may correspond to a convergence path in a multifocal lens. The convergence path in multifocal lenses is usually the main gradient path along which the optical power changes. Usually the convergence path is a substantially vertical central meridian, with possible deviation to account for small eye convergence for near vision tasks, for example, because close objects are usually viewed through the lower part of the lens. Accordingly, the convergence path may be the path the eye makes across the lens when looking at objects at different distances while lowering the gaze.

When mask 44 is located positioned accurately on lens 42, e.g., so that transparent stain 48 may be located substantially on the convergence path of lens 42, a patient may wear spectacles 40 and aim his gaze in the directions of plurality of objects of interest through different points along the transparent stain 48; this corresponds to different points along the convergence path of lens 42, while holding the head in a natural position for looking at the objects. In case the patient is able to see the objects clearly, he may conclude that the convergence path and the corresponding transparent stain 48 are located substantially in the correct position in front of his pupil, and therefore, for example, he may conclude that lens 42 is fitted correctly in spectacles frame 40. However, in the case the patient is not able to see the objects clearly, he may conclude that the convergence path and the corresponding transparent stain 48 are not located in the correct position in front of his pupil and therefore, he may conclude that lens 42 is not fitted correctly in spectacles frame 40.

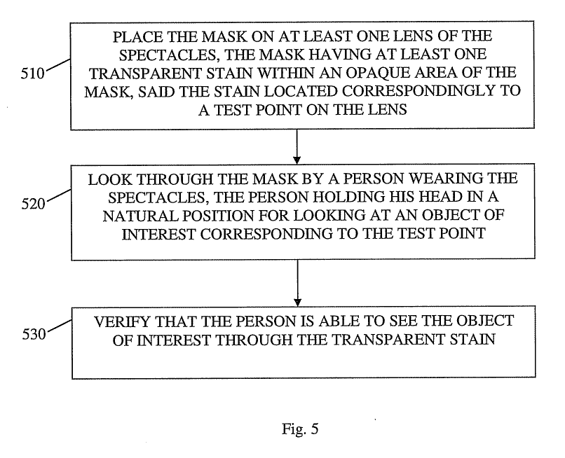

Reference is now made to FIG. 5, which is a flowchart illustrating a method for measuring and/or verifying the correct fitting of a lens in a spectacles frame according to some embodiments of the present invention. As shown in block 510, the method according to some embodiments of the present invention may include placing a mask on at least one lens installed in a spectacles frame. The mask may have at least one transparent stain, for example, within an opaque area of the mask. The stain may be located correspondingly to a test point on the lens. As shown in block 520, the method according to some embodiments of the present invention may include looking through the mask by a person wearing the spectacles, the person holding his head in a natural position for looking at an object of interest corresponding to the test point. As shown in block 530, the method according to some embodiments of the present invention may include verifying that the person is able to see the object of interest through the stain.

The mask and the method described above may be used also for measurement of the desirable position of points of interest on the lens relative to the frame. As described above in the background of the invention, the standard practice in the industry is to mark these points with marking pen on demo lenses installed in the chosen spectacles frame. However, by using embodiments of the present invention, the optician may apply mask 34 or 44, for example, on a demo lens. The patient may look through mask 34 or 44 and determine the correct position of stain 38 or 48 and therefore, for example, the correct position of the point of interest on the lens. The determination of the correct position of stain 38 or 48 may be done by looking through mask 34 or 44 at an object of interest corresponding to the examined points of interest. For example, for determination of a correct position of a fitting point, the patient may look at a far object through mask 34. If the patient sees the object in a satisfactory quality of vision, the patient may conclude that stain 38 is in the correct location on the lens, and therefore, that this is the correct location for the fitting point.

Some embodiments of the present invention may enable installation measurements and/or medical examinations. For example, a mask 34 according to some embodiments of the present invention may be used for measuring the distance between the lens and the eye, usually called back vertex distance. Additionally or alternatively, mask 34 may be used for measuring, for example, the eye length.

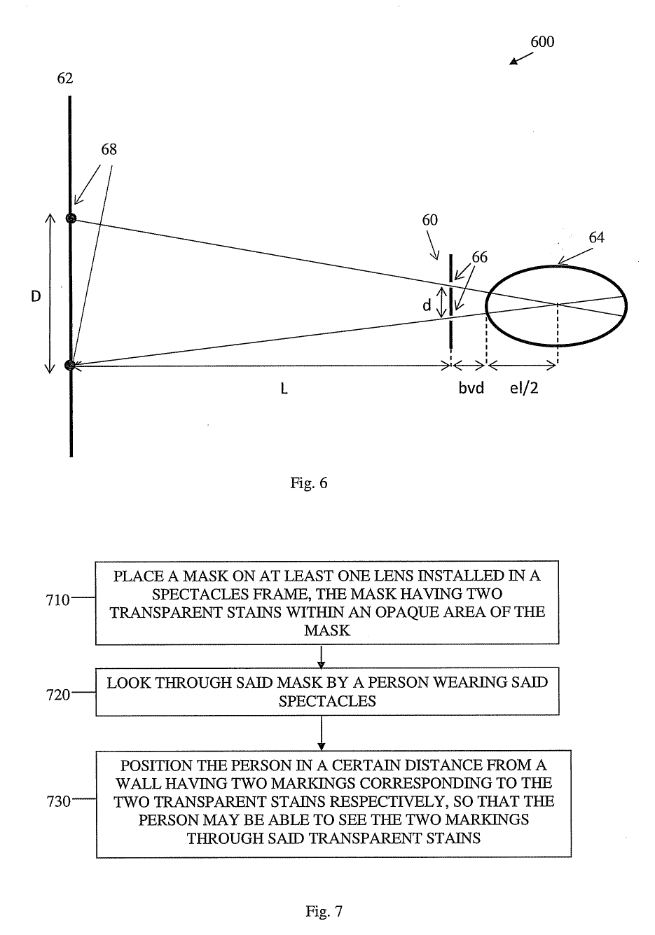

Reference is now made to FIG. 6, which is a schematic illustration of a system 600 for back vertex distance and/or eye length measurements according to some embodiments of the present invention. System 600 may include a mask 60, a wall 62 and an eye 64. Mask 60 may include two holes or transparent stains 66, for example, with distance d between them. Mask 60 may be installed on a spectacles frame (such as, for example, frame 30 of FIG. 3), which may be worn by a patient and may correspond, for example, to a specific lens designated to be installed on said frame. For example, stains 66 may correspond to points of interest on said lens. Wall 62 may include two markings 68, for example, with distance D between them, corresponding to stains 66. For example, when the patient is located in a specific distance L from wall 62, the patient may see markings 68 through stains 66, respectively. The sum of the back vertex distance (bvd) and half of the eye length (el) can be found based on the pre-measured distances L, D and d, for example, according to the formula bvd+0.5*el=L*d/(D−d). If the eye length is known, the back vertex distance may be calculated. The back vertex distance may correspond to the distance between the eye and the lens and/or the mask installed thereon.

In other embodiments of the present invention, if the back vertex distance is known, the eye length may be calculated, which may be required, for example, for medical and/or optometrical examinations/diagnosis. Additionally or alternatively, it is possible to measure the eye length by bringing the mask as close as possible to the eye, so that the back vertex distance is close to zero and can be neglected in the calculation.

Additionally, it is possible to measure the difference between the back vertex distance for two different masks (which may correspond, for example, to two different lenses), by performing the calculation of the sum of the back vertex distance (bvd) and half of the eye length (el) in two different measurements with corresponding two different masks on the same person.

Alternatively, it is possible to measure the difference between the eye length of two different persons, by performing the calculation of the sum of the back vertex distance (bvd) and half of the eye length (el) in two different measurements with the same mask and same L, D, d and bvd values, wherein the mask is worn by a different person in each measurement.

Reference is now made to FIG. 7, which is a flowchart illustrating a method for optometric measurements according to some embodiments of the present invention. As shown in block 710, the method may include, for example, placing a mask on at least one lens installed in a spectacles frame, the mask having two transparent stains within an opaque area of the mask. As shown in block 720, the method may include, for example, looking through said mask by a person wearing said spectacles. As shown in block 730, the method may include, for example, positioning the person in a certain distance from a wall having two markings corresponding to the two transparent stains respectively, so that the person may be able to see the two markings through said transparent stains, respectively.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for measuring and/or verifying the fitting of ophthalmic lenses in spectacles frame, the method comprising:
placing a mask on at least one lens installed in said spectacles frame, the mask having at least one transparent stain within an opaque area of the mask, said stain located correspondingly to a test point on said lens.

2. A method according to claim 1, further comprising looking through said mask by a person wearing said spectacles, the person holding his head in a natural position for looking at an object of interest corresponding to said test point.

3. A method according to claim 2, further comprising verifying that said person is able to see said object of interest through said stain.

4. A method according to claim 1, wherein the shape of said stain is one of a list comprising a spot, point, line or any other shape of interest.

5. A method according to claim 1, wherein said placing is by sticking said mask on the lens.

6. A method according to claim 5, wherein said sticking is by electrostatic effect.

7. A method according to claim 1, wherein said placing is such that pre-marked markings on the lens coincide with corresponding markings on the mask.

8. A device for measuring and/or verifying the fitting of ophthalmic lenses in spectacles frame, the device comprising:
a mask to be placed on at least one lens of said spectacles, the mask having at least one transparent stain within an opaque area of the mask, said stain located correspondingly to a test point on said lens.

9. A device according to claim 8, wherein the shape of said stain is one of a list comprising a spot, point, line or any other shape of interest.

10. A device according to claim 8, wherein said mask is a sticker.

11. A device according to claim 8, wherein said mask can be stick by electrostatic effect.

12. A device according to claim 8, wherein said mask has markings corresponding to pre-marked markings on the lens, so that when said pre-marked markings on the lens coincide with said corresponding markings on the mask when said mask is placed on the lens, the stain would coincide with said test point.

13. A system for performing optometrical measurements, the system comprising:
a mask to be placed on at least one lens of installed in a spectacles frame, the mask having at least two transparent stains within an opaque area of the mask;
a wall having two markings corresponding to said at least two transparent stains respectively, wherein a person wearing said spectacles frame is able to see said two markings through said transparent stains when positioned in a certain distance from said wall.

14. A method for performing optometrical measurements, the method comprising
placing a mask on at least one lens installed in a spectacles frame, the mask having at least two transparent stains within an opaque area of the mask;
looking through said mask by a person wearing said spectacles;
positioning said person in a certain distance from a wall having two markings corresponding to said at least two transparent stains respectively, wherein said person is able to see said two markings through said transparent stains when positioned in said certain distance.
